(19) United States
(12) Patent Application Publication
(10) Pub. No.: US 2003/0114871 A1
(43) Pub. Date: Jun. 19, 2003

(54) Medico-surgical apparatus

(76) Inventor: Christopher Stratton Turnbull, Hythe (GB)

Correspondence Address: LOUIS WOO, LAW OFFICE OF LOUIS WOO, 717 NORTH FAYETTE STREET, ALEXANDRIA, VA, 22314, US

(21) Appl. No.: 10/290,279

(22) Filed: Nov. 8, 2002

(30) Foreign Application Priority Data
Dec. 18, 2001 (GB) 0130156.3

(51) Int. Cl. A61B 17/34

(52) U.S. Cl. 606/167; 606/185; 606/190

(57) ABSTRACT

A percutaneous tracheostomy assembly comprises a dilator mounted on a needle. The needle has a hollow metal shaft with a pointed tip at one end and a hub at its opposite end. The dilator is of plastics with a narrow, flexible patient end portion, a tapering intermediate portion and a substantially rigid, wider rear portion. The tip of the needle projects from the patient end of the dilator when the needle hub abuts the rear end of the dilator so that the patient end of the assembly can be pushed into the trachea. The length of the patient end portion of the dilator is such that the tip of the needle is spaced from the posterior wall of the trachea when the tapered portion of the dilator abuts the surface of the skin. After insertion, the needle is replaced with a guidewire so that the dilator can be pushed in further to expand the opening.

### BACKGROUND OF THE INVENTION

This invention relates to medico-surgical apparatus.

One technique for performing a percutaneous tracheostomy involves a needle carrying a cannula. An initial cut is made with a scalpel through the skin of the neck and the needle is pushed through neck tissues and the anterior wall of the trachea so that the tip of the needle and the cannula locate in the trachea. The needle is then pulled out to leave the cannula in place. A guidewire is inserted through the cannula, which is then removed. A first dilator or pre-dilator is slid along the guidewire to expand the opening through the tissue slightly to between 4-5 mm, sufficient to enable a larger dilator to be inserted. The first dilator is typically 5 cm long, it is straight and relatively rigid. The opening is then expanded with the larger dilator or with subsequent, progressively larger dilators until it is large enough to receive a percutaneous tracheostomy tube.

One problem with this technique is that there is a risk the first dilator will be pushed in too far and contact the posterior wall of the trachea, causing damage. This problem is made worse because the cartilagenous anterior wall of the trachea presents a high resistance to penetration and, once the dilator has passed through this the resistance drops suddenly with the risk that dilator will move forwardly rapidly. Also, there is a greater risk of contact with the posterior wall of the trachea because the force applied by the dilator tends to push the anterior wall of the trachea towards the posterior wall, reducing the gap between them.

### BRIEF SUMMARY OF THE INVENTION

It is an object of the present invention to provide alternative medico-surgical apparatus.

According to one aspect of the present invention there is provided a tracheostomy dilator adapted for insertion through tissue into the trachea on a needle, the dilator having a bore extending therethrough to receive the needle, a first, patient end portion and a second portion extending rearwardly from the first portion, the first portion being flexible and of substantially constant external diameter, and the second portion being more rigid and tapering along at least a part of its length to a larger diameter towards its rear end.

The length of the first portion is preferably such that when the forward end of the second portion abuts the surface of the neck the tip of the first portion is located within the trachea spaced from the posterior wall. The first portion may be substantially 20 mm long and have an external diameter of substantially 2 mm. The second portion preferably tapers to a diameter of about 4.6 mm at its rear end and tapers along a distance of substantially 15 mm. The dilator preferably includes a rear end portion extending from the second portion and having a substantially constant external diameter. The dilator is preferably moulded of a plastics material.

According to another aspect of the present invention there is provided a tracheostomy assembly of a needle and a dilator removably mounted on the needle, the dilator having a bore extending therethrough along which the needle extends, a first, patient end portion and a second portion extending rearwardly from the first portion, the first portion being flexible and of substantially constant external diameter, and the second portion being more rigid and tapering along at least a part of its length to a larger diameter towards its rear end.

The assembly is preferably straight. The needle preferably has a pointed tip at its patient end and a hub at its rear end, the tip projecting beyond the patient end of the dilator and the hub engaging the rear end of the dilator. The assembly is preferably arranged such that when the forward end of the second portion of the dilator abuts the surface of the neck the tip of the needle is located within the trachea spaced from the posterior wall. The assembly may include a guidewire that is insertable in the dilator after removal of the needle.

A percutaneous tracheostomy assembly including a dilator according to the present invention, will now be described, by way of example, with reference to the accompanying drawing.

### BRIEF DESCRIPTION OF THE DRAWING

FIG. 1 is a cross-sectional side elevation view of the assembly; and

FIGS. 2 to 5 are partly sectional side elevation views showing the assembly and dilator in use.

### DETAILED DESCRIPTION OF THE PREFERRED EMBODIMENT

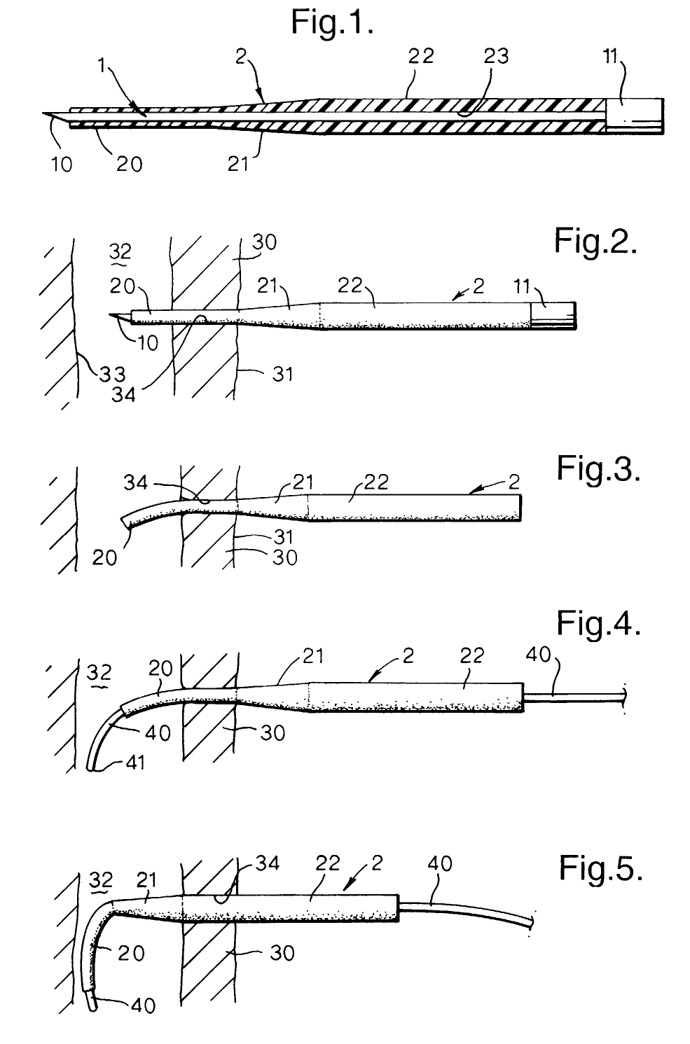

With reference first to FIG. 1, the assembly includes a needle 1 and a dilator 2.

The needle 1 is hollow and of metal, with a pointed penetrating tip 10 at one end and a moulded hub 11 at its opposite end.

The dilator 2 is tubular and of plastics material. The dilator has a flexible forward, patient end portion 20 that is about 20 mm long and has a constant external diameter of about 2 mm along its length. A second, intermediate portion 21 extends from the rear of the forward end portion 20 and is about 15 mm long. The intermediate portion 21 tapers outwardly along its length from a diameter of 2 mm at its forward end to a diameter of 4.6 mm at its rear end. This intermediate portion 21 is substantially rigid. The rear, machine end portion 22 of the dilator 2 has a constant diameter of 4.6 mm and extends for a distance of about 40 mm; this also is substantially rigid. A bore 23 extends coaxially along the entire length of the dilator 2.

The total length of the dilator 2 is such that, when fitted on the needle 1, the rear end of the dilator abuts the hub 11 of the needle and its tip 10 is exposed at the forward end of the dilator.

FIGS. 2 to 5 show how the assembly is used. First, as shown in FIG. 2, the assembly is pushed through the neck tissue 30, after having first made a cut through the skin 31, until the tip 10 of the needle 1 and the patient end of the dilator locate in the trachea 32. Penetration of the trachea could be detected in a conventional manner, such as by aspirating air with a syringe connected to the hub 11. The length of the patient end portion 20 of the dilator 2 is such that when the forward end of the intermediate portion 21 abuts the skin 31, the tip of the assembly is located in the trachea spaced from the posterior wall. The taper on the intermediate portion 21 increases the resistance to insertion of the assembly and helps reduce the risk of the assembly being inserted too far and damaging the posterior wall 33 of the trachea 32. Next, as shown in FIG. 3, the needle 1 is removed, leaving the dilator 2 in position. Then the user inserts a guidewire 40 through the bore 23 of the dilator 2, as shown in FIG. 4, so that its forward end 41 projects from the dilator within the trachea 32. The user then grips the rear end 22 of the dilator 2 and pushes it in along the guidewire 40, as shown in FIG. 5. As the intermediate portion 21 enters the neck tissue 30 it starts to expand the opening 34 through the tissue from 2 to 4.6 mm. The dilator 2 is pushed in until the intermediate portion 21 lies within the trachea 32 and the rear portion 22 is located in the neck tissue 30. This operation can be performed smoothly in a controlled manner because the tip of the dilator 2 has already penetrated the stiff cartilagenous front wall of the trachea 32. The flexible forward portion 20 of the dilator 2 is guided by the guidewire and prevents any damage to the posterior wall 33 of the trachea 32. The dilator 2 is then pulled out, leaving the guidewire 40 in position and subsequent dilators (not shown) are inserted along it in the usual way until the opening 34 to the trachea 32 is large enough to receive a percutaneous tracheostomy tube.

The present invention enables the risk of damage to the posterior wall of the trachea to be reduced. The invention also simplifies the formation of a tracheostomy by reducing the number of steps. Because a single component is used there is no need for a separate cannula to be removed before a pre-dilator can be inserted. The term “tracheostomy” is used to denote any opening providing access to the trachea including in the cricothyroid region.

The dilator of the invention could be modified for use with a pre-dilator by shortening the patient end tip of the dilator and reducing the maximum diameter of the intermediate portion to about 3 mm. Such an arrangement would still reduce the risk of damage to the posterior wall of the trachea by the subsequently-inserted pre-dilator because its entry would be made easier by the larger size opening.

The patient end portion 20 of the dilator 2 could be formed such that it bends preferentially in one direction so that this can be directed caudally. Preferential bending could be achieved by means of a stripe of a different material having a different hardness, or by altering the wall thickness.

What I claim is:

1. A tracheostomy dilator adapted for insertion through tissue of the neck into the trachea on a needle and comprising: a first, patient end portion, said first portion being flexible and of substantially constant external diameter; a second portion extending rearwardly from said first portion, said second portion being more rigid and tapering along at least a part of its length to a larger diameter towards a rear end; and a bore extending through the dilator along its length to receive said needle.

2. A dilator according to claim 1, wherein the length of said first portion is such that when a forward end of said second portion abuts a surface of the neck a tip of said first portion is located within the trachea spaced from a posterior wall of the trachea.

3. A dilator according to claim 1, wherein said first portion is substantially 20 mm long.

4. A dilator according to claim 1, wherein the external diameter of said first portion is substantially 2 mm.

5. A dilator according to claim 1, wherein said second portion tapers to a diameter of about 4.6 mm at its rear end.

6. A dilator according to claim 1, wherein said second portion tapers along a distance of substantially 15 mm.

7. A dilator according to claim 1, wherein said dilator includes a rear end portion extending from said second portion and having a substantially constant external diameter.

8. A dilator according to claim 1, wherein said dilator is moulded of a plastics material.

9. A tracheostomy dilator adapted for insertion through tissue of the neck into the trachea on a needle and comprising: a first, patient end portion, said first portion being flexible and of substantially constant external diameter; a second portion extending rearwardly from said first portion, said second portion being more rigid and tapering along at least a part of its length to a larger diameter of about 4.6 mm towards its rear end; a third, rear end portion of substantially constant external diameter extending from the rear of said second portion; and a bore extending through the dilator along its length to receive said needle, and wherein the length of said first portion is such that when a forward end of said second portion abuts the surface of the neck the tip of said first portion is located within the trachea spaced from the posterior wall of the trachea.

10. A tracheostomy assembly of a needle and a dilator removably mounted on said needle, wherein said dilator comprises: a first, patient end portion, said first portion being flexible and of substantially constant external diameter; a second portion extending rearwardly from said first portion, said second portion being more rigid and tapering along at least a part of its length to a larger diameter towards a rear end; and a bore extending through said dilator along its length within which said needle extends.

11. An assembly according to claim 10, wherein said assembly is straight.

12. An assembly according to claim 10, wherein said needle has a pointed tip at its patient end and a hub at its rear end, wherein said tip projects beyond the patient end of said dilator, and wherein said hub engages the rear end of said dilator.

13. An assembly according to claim 10, wherein said assembly is arranged such that when a forward end of said second portion of the dilator abuts the surface of the neck the tip of said needle is located within the trachea spaced from the posterior wall.

14. An assembly according to claim 10 including a guidewire insertable in said dilator after removal of said needle.

15. A tracheostomy assembly of a metal needle and a plastics dilator removably mounted on said needle, wherein said dilator comprises: a first, patient end portion, said first portion being flexible and of substantially constant external diameter; a second portion extending rearwardly from said first portion, said second portion being more rigid and tapering along at least a part of its length to a larger diameter towards a rear end; and a bore extending through said dilator along its length within which said needle extends, wherein said needle has a hub at its rear end engaging a rear end of said dilator, and wherein said needle has a pointed tip projecting from a patient end of said dilator such that a patient end of the assembly can be inserted into the trachea and said needle removed from said dilator leaving said dilator in position with its first portion located in the trachea, such as to enable a guidewire to be inserted into the trachea along said bore of said dilator and such that said second portion of the dilator can be inserted to expand tissue by sliding along said guidewire.

16. A method of performing a percutaneous tracheostomy comprising: providing an assembly of a rigid needle and a dilator, said needle having a pointed tip exposed at a patient end of the assembly and said dilator extending along said needle; inserting said patient end of said assembly through neck tissue until the forward end of both said needle and said dilator are located within the trachea; removing said needle to leave said dilator in position; inserting a guidewire through said dilator; further inserting said dilator so that a tapered portion on said dilator expands said neck tissue; and withdrawing said dilator for subsequent insertion of a tracheostomy tube.
